(19) United States
(12) Patent Application Publication
(10) Pub. No.: US 2022/0040092 A1
(43) Pub. Date: Feb. 10, 2022

(54) Ready to use injectable formulations of Micafungin Sodium

(71) Applicant: RK Pharma Solutions LLC, Piscataway, NJ (US)

(72) Inventors: Ravishanker Kovi, Monroe Township, NJ (US); Raghu Kasu, Monmouth Jct, NJ (US); Jayaraman Kannappan, Vadodara (IN); AJEY REDDY, Bangalore (IN); SANJAY THAKOR, Vadodara (IN); KAPISH KAPOOR, Madhya Pradesh (IN)

(21) Appl. No.: 17/355,876

(22) Filed: Jun. 23, 2021

Related U.S. Application Data
(60) Provisional application No. 63/045,223, filed on Jun. 29, 2020.
(60) Provisional application No. 63/045,054, filed on Jun. 27, 2020.

(51) Int. Cl. A61K 9/00; A61K 38/12; A61K 47/02; A61K 47/26; A61K 47/12

(52) U.S. Cl. CPC A61K 9/0019; A61K 38/12; A61K 47/12; A61K 47/26; A61K 47/02

(57) ABSTRACT

The present application provides method for formulating and corresponding a ready-to-use liquid injectable parenteral pharmaceutical composition, comprising Micafungin sodium and a first excipient and/or pharmaceutically acceptable second excipient.

### FIELD OF THE INVENTION

The present application relates to stable pharmaceutical compositions of micafungin or a pharmaceutically acceptable salt thereof suitable for parenteral administration. In particular, the present application relates to a ready to use Micafungin sodium formulation having a stabilizer which controls the impurity. The application also relates to use of this formulation in treating fungal infection.

### BACKGROUND OF THE INVENTION

Micafungin for injection, commercially available under the trade name MYCAMINE®, is a sterile, lyophilized formulation containing Micafungin sodium. Micafungin sodium is furthermore known as FK-463. Micafungin sodium is a semisynthetic lipopeptide (echinocandin) synthesized by a chemical modification of a fermentation product of Coleophoma empetri F-11899. Micafungin inhibits the synthesis of 1, 3-beta-D-glucan, an integral component of the fungal cell wall.

Marketed single-use vial contains 50 mg or 100 mg micafungin sodium, 200 mg lactose, with citric acid and/or sodium hydroxide (used for pH adjustment). Mycamine must be diluted with 0.9% Sodium Chloride Injection, USP, or 5% Dextrose Injection, USP. Following reconstitution with 0.9% Sodium Chloride Injection, USP, the resulting pH of the solution is between 5-7.

Micafungin sodium is chemically designated as:

{5-[(1S,2S)-2-[(3S,6S,9S,11R,15S,18S,20R,21R,24S,25S,26S)-3-[(1R)-2-carbamoyl-1-hydroxyethyl]-11,20,21,25-tetrahydroxy-15-[(1R)-1-hydroxyethyl]-26-methyl-2,5,8,14,17,23-hexaoxo-18-[(4-{5-[4-(pentyloxy)phenyl]-1,2-oxazol-3-yl)benzene)amido]-1,4,7,13,16,22-hexaazatricyclo[22.3.0.0.0^{9,13}]heptacosan-6-yl]-1,2-dihydroxyethyl]-2-hydroxyphenyl} oxidanesulfonic acid. The molecular formula is C₅₆H₇₀NaN₉O₂₃S and the formula weight is 1292.26.

The chemical structure of micafungin sodium is:

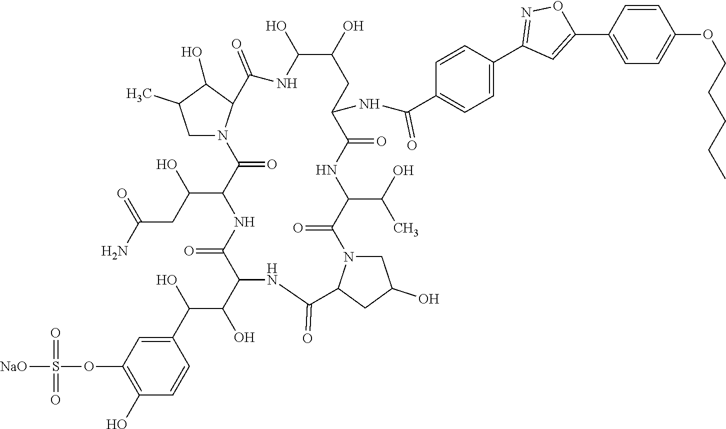

Micafungin sodium is a light-sensitive, hygroscopic white powder that is freely soluble in water, isotonic sodium chloride solution, N,N-dimethylformamide and dimethylsulfoxide. Micafungin is slightly soluble in methyl alcohol, and practically insoluble in acetonitrile, ethyl alcohol (95%), acetone, diethyl ether and n-hexane.

Micafungin is indicated for the treatment of candidemia, acute disseminated candidiasis, Candida peritonitis, abscesses and oesophageal candidiasis. Micafungin has been approved for the prophylaxis of Candida infections in patients undergoing hematopoietic stem cell transplantation (HSCT).

Micafungin works by way of concentration-dependent inhibition of 1,3-beta-D-glucan synthase resulting in reduced formation of 1,3-beta-D-glucan, which is an essential polysaccharide comprising one-third of the majority of Candida spp. cell walls. This decreased glucan production leads to osmotic instability and thus cellular lysis.

Micafungin and the preparation thereof are discussed in the U.S. Pat. No. 6,107,458, which is incorporated herein by reference. U.S. Pat. No. 6,774,104, which is incorporated herein by reference, discusses stable lyophilized compositions of micafungin and disaccharide such as lactose, maltose and sucrose. U.S. Pat. No. 7,112,565, which is incorporated herein by reference, discusses formulating stable lyophilized compositions of micafungin with disaccharide, polysaccharide or sodium chloride.

PCT International Pub No. WO/2012/103802, which is incorporated herein by reference, discusses the liquid pharmaceutical compositions of micafungin in aqueous solvent using at least one stabilizer such as monosaccharide, disaccharide or polysaccharide, or combinations thereof; preferably, lactose, sucrose, maltose, trehalose or combinations thereof.

U.S. Pat. No. 8,980,827, which is incorporated herein by reference, discusses the use of Trehalose as stabilizing agent to prepare Micafungin formulation. US Patent Pub. No. 20190151241, which is incorporated herein by reference, discusses the composition comprising micafungin, one or more non-aqueous solvents such as polar aprotic solvent and optionally water. Another application JP 2019089725, which is incorporated herein by reference, discusses the use of gluconic acid, inorganic salts of gluconic acid, and inositol as stabilising agent in the preparation of Micafungin formulation. Various CN application 103330932, 103330933 and 105106933, which are incorporated herein by reference, discuss the formulation product of Micafungin.

US Patent Pub. No. 20180169180, which is incorporated herein by reference, discusses the use of buffering agent and tonicity adjusting agent for preparation of aqueous Micafungin sodium.

Uncharacteristically, it was observed when ready to use solutions are prepared by use of Micafungin sodium and lactose as stabilising agent alone, an unknown impurity at 0.93 RRT is found, which affects the overall purity of Micafungin formulation.

In addition, Micafungin and the salts are generally unstable to light, heat, humidity, acid, and the like. Therefore, there is need to develop a pharmaceutical composition for stabilizing the compound and the salts thereof.

Moreover, the prescribing information teaches that MYCAMINE® should only be stored for a maximum of 24 hours after compounding for infusion. Due to that it has to be use within a mention time. To avoid such a drawback, we have found stable solution containing Micafungin sodium in infusion bag and vial, which is stable more than 3 months at 2-8° C.

The currently marketed form of Micafungin sodium for injection is costly to manufacture and inconvenient to use because it is not in a ready-to-use format. Therefore, an aqueous and ready-to-use Micafungin sodium solution formulation is highly desirable. In particular, the ready-to-use, injectable formulations described herein are stable, allow medical personals to use prepared containers containing an injectable formulation off the shelf without additional preparation, avoid potential contamination problems, and eliminate dosage errors.

### SUMMARY OF THE INVENTION

Embodiments in accordance with the present disclosure provide a stable, ready-to-use injectable Micafungin sodium solution which is preferably easy to administer without need of any reconstitution step and has a improve solubility, stability and safety profile.

In one or more embodiments there is provided a parenteral formulation of Micafungin sodium. Formulation(s) prepared by this application are preferably stored in infusion bag or vial.

Specifically, one or more embodiments of the present application provides a ready-to-use liquid injectable parenteral pharmaceutical composition, comprising Micafungin sodium and a first excipient, wherein the first excipient is one or more selected from the group consisting of polysaccharides, disaccharides and a combination thereof. Preferably, the first excipient according to the present invention can be selected from the group consisting of polysaccharides, such as one or more of lactose, maltose and sucrose. The first excipient can preferably be lactose.

The main effect of the first excipient in the pharmaceutical composition of the present invention is to improve the stability of the active ingredient micafungin in the solubilisation process.

In a preferred embodiment, the composition of the present invention also comprises a second excipient that mainly plays a protective role in storage process. The second excipient can be selected from the group consisting of sodium metabisulfite, Sodium thiosulphate, methionine, Ascorbic acid, Butylated Hydroxytoluene,

In one of the other embodiments, Micafungin formulations are also prepared without using first excipient in formulation.

In still further embodiments provided are ready-to-use liquid parenteral formulations including Micafungin sodium, one or more pharmaceutically acceptable stabilizing agents and solvents, co-solvents, and/or solubilising agent.

The storage-stable, ready-to-use, injectable compositions of the present invention are useful as antifungal.

The details of one or more embodiments of the invention are set forth in the description below. Other features, objects and advantages of the invention will be apparent from the description.

### DETAILED DESCRIPTION OF THE PREFERRED EMBODIMENTS

The present inventive concepts will now be described more fully hereinafter with reference to the accompanying examples and experiments, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, “Micafungin” refers to Micafungin and the pharmaceutically acceptable salts, solvates, hydrates and anhydrous forms thereof.

As used here in “ready-to-use” when used in connection with a Micafungin sodium formulation refers to a liquid formulation that includes Micafungin sodium in dissolved or solubilized form and/or is intended to be used as such or upon further dilution in intravenous diluents.

As used herein, and unless otherwise specified, the term “storage-stable” refers to any liquid Micafungin sodium-containing composition or formulation having sufficient physical and chemical stability to allow storage at a convenient temperature above the freezing point of the composition or formulation for a commercially reasonable period of time. Formulations prepared by this invention are stored in infusion bag or vial. The phrase “physical stability” refers to maintenance of colour or colourless state, dissolved oxygen level, head space oxygen level and particulate matter and the phrase “chemical stability” relates to formation of drug-related impurities in terms of total impurities, single maximum individual impurity, or maximum individual unknown impurity. For pharmaceutical products, stability is required for commercially relevant times after manufacturing, such as for about 6, 12, 18, 24, or 36 months, during which time a product is kept in its original packaging under specified storage conditions.

As used herein, “relative retention time” (“RRT”) is calculated by dividing the retention time of the peak of interest by the retention time of the main peak. Any peak with an RRT<1 elutes before the main peak, and any peak with an RRT>1 elutes after the main peak.

As used herein, and unless otherwise specified, the term “about” means an acceptable error for a particular value as determined by one of ordinary skill in the art, which depends in part on how the value is measured or determined. In certain embodiments, the term about means within 10%, 9%, 8%, 7%, 6%, 5%, 4%, 3%,2%, 1%, 0.5%, 0.1%, or 0.05% of a given value or range.

Approved Micafungin composition contains Micafungin sodium, lactose, citric acid and sodium hydroxide for pH adjustment. When such a formulation was prepared as a ready to use solution it was uncharacteristically observed an impurity at 0.93 RRT. This impurity decreases the overall purity required for drug approval. Because of one of this reason, it is required to develop a ready to use formulation which is stable for at least 6 months.

In one embodiment, the present application provides a ready-to-use liquid injectable parenteral pharmaceutical composition comprising Micafungin sodium and a first excipient, wherein the first excipient is one or more selected from the group consisting of polysaccharides, disaccharides and a combination thereof.

Preferably, the first excipient can be selected from the group consisting of polysaccharides, such as one or more of lactose, maltose and sucrose. The first excipient can be preferably lactose.

In another embodiment, the present application provides a ready-to-use liquid injectable parenteral pharmaceutical composition comprising Micafungin sodium and second excipient. Second excipient mainly plays a protective role in storage process.

In one of the other embodiments, Micafungin formulations are also prepared without using first excipient in formulation. Herein the second excipient controls the impurity in the formulation.

The second excipient can be selected from the group consisting of sodium metabisulfite, Sodium thiosulphate, methionine, Ascorbic acid, Butylated Hydroxytoluene,

Pharmaceutically acceptable additional excipients or adjuvants include but are not limited to one or more preservatives, polymers, pH adjusting agents, surfactants, chelating agents, dispersing agents, binding agents, tonicity modifying agents and antioxidants.

Examples of pharmaceutically acceptable pH adjusting agents include but are not limited to sodium hydroxide, hydrochloric acid, acetic acid, phosphoric acid, succinic acid, potassium hydroxide, ammonium hydroxide, etc. and combinations thereof. This pH adjusting agents helps to maintain the pH between 5 to 6.

Pharmaceutically acceptable vehicles include but are not limited to 0.9% NaCl, sterile water for injection, dextrose, lactated ringer solution and combinations thereof.

The formulations according to the present invention may be in the form of clear injectable solution, suspension or emulsion.

In some embodiments the storage-stable ready-to-use injectable formulation may have a concentration of Micafungin of less than 50 mg/ml. In other embodiments the injectable formulation may have a concentration of Micafungin of from about 1 mg/ml to about 42 mg/ml. In another embodiment the injectable formulation may have a concentration of Micafungin of from about 10 mg/ml to about 30 mg/ml. In other embodiments the injectable formulation may have a concentration of Micafungin of about 20 mg/ml.

The storage-stable, ready-to-use injectable Micafungin sodium-containing formulations disclosed herein do not require any additional reconstitution step at the time of administration.

Formulations in accordance with the present disclosure have a controlled impurity profile suitable for regulatory approval at various storage conditions. The storage-stable ready-to-use Micafungin sodium formulations may be stored at about 0° C. to about 40° C. The storage-stable, ready-to-use Micafungin sodium formulations for injection may retain at least 90% of the potency of Micafungin sodium after storage for six months at about 0° C. to about 40° C. temperature and 60% relative humidity.

The storage-stable, ready-to-use, injectable formulations may be formulated to provide single or multiple dosage administration. The single dosage formulation may be packaged in an ampoule, a vial, or a syringe. Multiple dosage formulations may be packaged in a vial. Multiple dosage formulations may preferably include at least one preservative.

The formulations have a pH value from about 2 to about 10. In some embodiments the pH range is from about 4 to about 8. In still other embodiments the pH range is from about 5 to about 6.

Storage-stable ready-to-use, injectable formulations disclosed herein contain Micafungin sodium having a purity of from about 97% to about 102%. In some embodiments the formulation contains Micafungin sodium having a purity of from about 90% to about 110%. In some embodiments the formulation contains Micafungin sodium having a purity of about 100%.

While the foregoing invention has been described in some detail for purposes of clarity and understanding, it will be appreciated by one skilled in the art, from a reading of the disclosure, that various changes in form and detail can be made without departing from the true scope of the invention.

### EXAMPLES

The following examples are for the illustration only and are not intended in any way to limit the scope of the present invention.

### Example 1

TABLE 1

| Ingredients (Trial 1) | Qty/vial |
|---|---|
| Micafungin sodium | 20 mg |
| Sodium metabisulfite | 0.9 mg |
| 1N sodium hydroxide | q.s. |
| solution (for pH adjustment) | |

Micafungin sodium 20 mg and sodium metabisulfite 0.9 mg were dissolved in 0.8 ml sterile water for injection. pH was adjusted to 5.5 using 1 M Sodium hydroxide solution, as required. Volume was made up to 1 ml with sterile water for injection. The vials were stored at refrigeration condition. Stability data is summarized in Table 1A.

TABLE 1A

| Stability at 2-8° C. | API | 1 month | 3 month |
|---|---|---|---|
| Purity | 99.25 | 99.01 | 98.7 |
| Imp at RRT 0.21 | ND | ND | 0.02 |
| Imp at RRT 0.83 | ND | 0.02 | 0.06 |
| Imp at RRT 0.93 | 0.64 | 0.51 | 0.52 |
| Imp at RRT 1.02 | 0.24 | 0.25 | 0.32 |
| Imp. E | 0.01 | 0.04 | 0.14 |
| Max. Unk Imp. | 0.04 | 0.04 | 0.05 |

### Example 2

TABLE 2

| Ingredients (Trial 2) | Qty/vial |
|---|---|
| Micafungin sodium | 20 mg |
| Sodium metabisulfite | 0.5 mg |
| 1N sodium hydroxide | q.s. |
| solution (for pH adjustment) | |

Micafungin sodium 20 mg and sodium metabisulfite 0.5 mg were dissolved in 0.8 ml sterile water for injection. pH was adjusted to 5.5 using 1 M Sodium hydroxide solution, as required. Volume was made up to 1 ml with sterile water for injection. The vials were stored at refrigeration condition. Stability data is summarized in Table 2A.

TABLE 2A

| Stability at 2-8° C. | API | 1 month | 3 month |
|---|---|---|---|
| Purity | 99.25 | 99.03 | 98.7 |
| Imp at RRT 0.21 | ND | ND | 0.02 |
| Imp at RRT 0.83 | ND | 0.03 | 0.06 |
| Imp at RRT 0.93 | 0.64 | 0.50 | 0.50 |
| Imp at RRT 1.02 | 0.24 | 0.25 | 0.29 |
| Imp. E | 0.01 | 0.04 | 0.10 |
| Max. Unk Imp. | 0.04 | 0.04 | 0.06 |

### Example 3

TABLE 3

| Ingredients (Trial 3) | Qty/vial |
|---|---|
| Micafungin sodium | 20 mg |
| Sodium metabisulfite | 1.5 mg |
| 1N sodium hydroxide | q.s. |
| solution (for pH adjustment) | |

Micafungin sodium 20 mg and sodium metabisulfite 1.5 mg were dissolved in 0.8 ml sterile water for injection. pH was adjusted to 5.5 using 1M Sodium hydroxide solution, as required. Volume was made up to 1 ml with sterile water for injection. The vials were stored at refrigeration condition. Stability data is summarized in Table 3A.

TABLE 3A

| Stability at 2-8° C. | API | 0 Day | 2 month | 3 month |
|---|---|---|---|---|
| Purity | 98.97 | 98.71% | 99.03 | 98.7 |
| Imp at RRT 0.21 | ND | ND | ND | ND |
| Imp at RRT 0.83 | ND | 0.35% | 0.03 | 0.03 |
| Imp at RRT 0.93 | 0.58 | 0.32% | 0.38 | 0.25 |
| Imp at RRT 1.02 | 0.24 | 0.43% | 0.42 | 0.45 |
| Imp. E | 0.02 | 0.04% | 0.07 | 0.08 |
| Max. Unk Imp. | 1.03 | 1.28% | 1.43 | 1.46 |

### Example A

TABLE 4

| Ingredients (Trial 4) | Qty/vial | |
|---|---|---|
| Micafungin sodium | 20 | mg |
| Lactose monohydrate | 20 | mg |
| Glacial acetic acid | 0.0015 | ml |
| Sodium metabisulfite | 9.0 | mg |

| | |
|---|---|
| 1N sodium hydroxide | q.s. |
| solution (for pH adjustment) | |

Micafungin sodium 20 mg, Lactose monohydrate 20 mg, Glacial acetic acid 0.0015 ml and sodium metabisulfite 9 mg were dissolved in 0.8 ml sterile water for injection. pH was adjusted to 5.5 using 1M Sodium hydroxide solution, as required. Volume was made up to 1 ml with sterile water for injection. The vials were stored at refrigeration condition. Stability data is summarized in Table 5A.

TABLE 5A

| Stability at 2-8° C. | API | 0 Day | 1 month | 4 month | 5 month |
|---|---|---|---|---|---|
| Purity | 98.97 | 98.94 | 98.9 | 98.54 | 98.55 |
| Imp at RRT 0.21 | ND | ND | ND | ND | ND |
| Imp at RRT 0.83 | ND | ND | 0.16 | 0.27 | 0.28 |
| Imp at RRT 0.93 | 0.58 | 0.58 | 0.42 | 0.36 | 0.37 |
| Imp at RRT 1.02 | 0.24 | 0.24 | 0.24 | 0.30 | 0.31 |
| Imp. E | 0.02 | 0.02 | 0.02 | 0.04 | 0.04 |
| Max. Unk Imp. | 1.03 | 1.03 | 1.10 | 1.75 | 1.51 |

### Example 5

TABLE 5

| Ingredients (Trial 5) | Qty/vial | |
|---|---|---|
| Micafungin sodium | 20 | mg |
| Lactose monohydrate | 20 | mg |
| Glacial acetic acid | 0.0015 | ml |

| | |
|---|---|
| 1N sodium hydroxide | q.s. |
| solution (for pH adjustment) | |

Micafungin sodium 20 mg, Lactose monohydrate 20 mg, Glacial acetic acid 0.0015 ml were dissolved in 0.8 ml sterile water for injection. pH was adjusted to 5.5 using 1M Sodium hydroxide solution, as required. Volume was made up to 1 ml with sterile water for injection. The vials were stored at refrigeration condition. Stability data is summarized in Table 5A.

TABLE 5A

| Stability at 2-8° C. | API | 0 Day | 1 month | 4 month | 5 month |
|---|---|---|---|---|---|
| Purity | 98.97 | 98.97 | 98.88 | 98.65 | 98.62 |
| Imp at RRT 0.21 | ND | ND | ND | ND | ND |
| Imp at RRT 0.83 | ND | 0.01 | ND | ND | ND |
| Imp at RRT 0.93 | 0.58 | 0.63 | 0.63 | 0.66 | 0.66 |
| Imp at RRT 1.02 | 0.24 | 0.25 | 0.25 | 0.30 | 0.32 |
| Imp. E | 0.02 | 0.04 | 0.04 | 0.05 | 0.05 |
| Max. Unk Imp. | 1.03 | 1.12 | 1.12 | 1.35 | 1.39 |

What is claimed is:

1. A ready-to-use liquid injectable parenteral formulation, comprising micafungin sodium and at least a first pharmaceutically acceptable first excipient.

2. The formulation of claim 1, wherein the pharmaceutically acceptable first excipient is selected from the group consisting of polysaccharides, disaccharides, and a combination thereof.

3. The formulation of claim 1, wherein the first excipient comprises a polysaccharide selected from a group consisting of lactose, maltose, and sucrose.

4. The formulation of claim 1, comprising a second pharmaceutically acceptable excipient selected from a group consisting of sodium metabisulfite, sodium thiosulfate, methionine, ascorbic acid, and butylated hydroxytoluene.

5. The formulation of claim 1, wherein the formulation further comprises one or more additional pharmaceutically acceptable excipients selected from a group consisting of one or more preservatives, polymers, pH adjusting agents, surfactants, chelating agents, dispersing agents, binding agents, tonicity modifying agents, and antioxidants.

6. The formulation of claim 1, wherein the formulation further comprises at least one pharmaceutically acceptable pH adjusting agent selected from a group consisting of sodium hydroxide, hydrochloric acid, acetic acid, phosphoric acid, succinic acid, potassium hydroxide, and ammonium hydroxide.

7. The formulation of claim 1, wherein the formulation further comprises at least one pharmaceutically acceptable vehicle selected from a group consisting of 0.9% NaCl, sterile water for injection, dextrose, and lactated ringer solution.

8. The formulation of claim 1, wherein formulation may be in the form of clear injectable solution, suspension, or emulsion.

9. The formulation of claim 1, having a concentration of Micafungin of less than 50 mg/ml.

10. The formulation of claim 1, wherein at least 90% potency of the micafungin sodium is retained after storage for 6 months at about 0° C. to about 40° C. temperature and 60% relative humidity.
